(19) United States
(12) Patent Application Publication
(10) Pub. No.: US 2016/0321962 A1
(43) Pub. Date: Nov. 3, 2016

(54) LABELING SYSTEM FOR IDENTIFICATION OF AN OBJECT

(71) Applicant: NK Medical Innvovations LLC, Redding, CT (US)

(72) Inventors: Nadia Koltchine, Redding, CT (US); Vladimir Koltchine, Redding, CT (US)

(73) Assignee: NK Medical Innovations LLC, Redding, CT (US)

(21) Appl. No.: 15/210,053

(22) Filed: Jul. 14, 2016

Related U.S. Application Data
(62) Division of application No. 14/817,917, filed on Aug. 4, 2015.
(60) Provisional application No. 62/114,353, filed on Feb. 10, 2015.

(51) Int. Cl. G09F 3/10; A61M 5/31; G09F 3/00

(52) U.S. Cl. CPC G09F 3/10; G09F 3/0297; A61M 5/31; G09F 2003/0208

(57) ABSTRACT

A labeling system includes one or more labels, each having a front side and a back side. The front side includes a first indicia of an object such as a medication. The front side and/or the back side includes a second indicia of the object, such as a matrix or linear bar code. The back side has an adhesive on a portion thereof opposite the first indicia. The back side also has an area free of adhesive. The area free of adhesive is aligned with the second indicia.

### CROSS REFERENCE TO RELATED APPLICATIONS

This application is a Divisional Application of U.S. patent application Ser. No. 14/817,917, filed on Aug. 4, 2015, which claims the priority to provisional patent application No. 62/114,353 filed Feb. 10, 2015, the entirety of which is incorporated herein by reference.

### TECHNICAL FIELD

This invention relates to a labeling system for identification of an object such as a medication and more particularly to a labeling system having labels that have indicia of the object disposed thereon in a scanable configuration.

### BACKGROUND

Drug substitution and incorrect dose errors in the operating room and in the hospital setting in general result in significant morbidity and mortality among patients so subjected. It is estimated that a drug substitution or incorrect dose error occurs in 1 out of every 140 procedures. It is further estimated that drug substitution and incorrect dose errors cost the average hospital approximately $2.8 million annually.

The Joint Commission on Accreditation of Healthcare Organizations (JCAHO) has identified drug labeling improvement as a way to reduce errors in the operating room, and has enacted regulations to effectuate such a change. Specifically, JCAHO regulations provide that all syringes must be labeled with (1) the name of the drug therein contained, (2) the concentration of the drug therein contained, (3) the date and time that the syringe was prepared, (4) the name of the person who prepared the drug, and (5) the expiration date and time the drug was administered.

Currently, all labels are color coded for easy recognition. For example, blue labels are used to indicate narcotics and other pain relieving medications, red labels are used to indicate paralyzing agents, and white labels are either left blank (i.e., to allow for manual entry of JCAHO required information) or used to indicate new or infrequently used medications.

There are currently a small number of machines on the market designed for printing labels that comply with JCAHO standards. However, these current machines are expensive, especially compared to the traditional method of writing the required information on a syringe label by hand. The labels produced by the current machines take longer to print than the traditional method, which becomes a problem when a patient must be administered multiple medications in a short period of time. The labels produced by the current machines require maintenance, and are difficult to clean if contaminated, e.g., with a patient's blood or with spilled medication. Further, hospitals that make use of the current machines still have to keep pre-printed labels on hand in case of a broken machine or a power outage. As a result, handwriting the JCAHO mandated information on syringe labels remains the primary form of drug preparation.

However, handwriting the JCAHO mandated information on syringe labels is difficult and time consuming. In a rapid workflow environment such as the operating room, wherein quick decisions and quick actions are required, handwriting the JCAHO mandated information on syringe labels is not ideal. Additionally, writing on the curved surface of a syringe by hand—especially when the syringe on which one must write is a small or low volume syringe—is difficult, and such writing is often difficult to decipher quickly.

As shown in FIG. 1A, a first syringe 100 has a label 110 with a small sized matrix barcode such as a Quick Response code (hereinafter “QR code®”) disposed thereon. QR Code® is a registered trademark of Denso Wave, Inc. of Tokyo, Japan. A QR scanner would be unable to analyze the information on the QR because the data pixels or “QR modules” are too small for the scanner to decipher accurately. Therefore, this QR code would not be scannable.

As shown in FIG. 1B, a second syringe 100′ has a label 110′ adhered to a curved surface of the second syringe 100′ such that both ends of the label 110′ wrap around the syringe 100′. The QR code printed on the label 110′ also wraps around the curved surface of the syringe 100′ such that portions of the QR code are not visible to the QR scanner. Therefore, the QR code on the label 110′ would not be scannable. This effect would be more prominent if the label would be placed on a small diameter, low volume syringe.

As shown in FIGS. 2A and 2B, there are multiple forms of medication labels for syringes currently available on the market. One such form is the Precut Roll Form 200 wherein a plurality of self-adhesive labels 210 with indicia of medication 214 printed on each label are positioned apart from one another on a release substrate 212. The Precut Roll Form 200 can be dispensed from a suitable roll rack that contains a plurality of the rolls 200. Another form is a Tape Roll Form 200′ including a continuous length of self-adhesive label tape 210′ with indicia of medication 214′ printed at predetermined intervals.

A system of labeling syringes that is consistent with JCAHO regulations and the use of which is fast and accurate has long been sought in the art.

### SUMMARY

In one aspect, the present invention resides in a labeling system for identification of an object such as a medication, a tube, a wire or a branch of a plant. The labeling system includes one or more labels, each having a front side and a back side. The front side includes a first indicia of the object, such as the name and dose of the medication. The front side and/or the back side includes a second indicia of the object, such as a matrix or linear bar code. The back side has an adhesive on a portion thereof opposite the first indicia. The back side also has an area free of adhesive. The area free of adhesive is aligned with the second indicia.

In another aspect, the present invention resides in a labeling system for identification of an object such as a medication, a tube, a wire or a branch of a plant. The labeling system includes one or more labels having a front side and a back side. The front side includes a first indicia (e.g., name and dose of the medication) of the medication and two or more second indicia (e.g., matrix or linear bar code) of the object positioned adjacent to one another. The back side has an adhesive thereon.

In another aspect the present invention resides in a labeling system for identification of an object such as a medication, a tube, a wire or a branch of a plant. The labeling system includes one or more labels having a front side and a back side. The front side includes a first indicia (e.g., name and dose of the medication) of the object. The front side and/or the back side has an extension segment extending therefrom. The extension segment has one or more second indicia (e.g., matrix or linear bar code) of the object positioned thereon. The back side has an adhesive thereon. The labeling system includes a plurality of the labels connected to one another in a continuous tape configuration. The continuous tape configuration is wound in a roll configuration. A free end of the tape defines a dispensing edge. The extension segment has a free end facing away from the dispensing edge.

### BRIEF DESCRIPTION OF THE DRAWINGS

FIG. 1A is a sketch of a syringe having a relatively small a prior art coded label adhered thereto;

FIG. 1B is a sketch of a syringe having a relatively large a prior art coded label adhered thereto;

FIG. 2A is a sketch of a roll of prior art coded labels in a pre-cut roll configuration;

FIG. 2B is a sketch of a roll of prior art coded labels in a tape roll configuration;

FIG. 3A is a schematic drawing of a front side of a label of the present invention having a code on the front side of the label;

FIG. 3B is a schematic drawing of a back side of a label of FIG. 3A;

FIG. 3C is a front view of a strip of five of the labels of FIG. 3A;

FIG. 3D is a back view of the strip of labels of FIG. 3C;

FIG. 4A is a schematic view of a label of the FIG. 3A illustrating one embodiment for placement of a code;

FIG. 4B is a schematic view of a label of the FIG. 3A illustrating another embodiment for placement of a code;

FIG. 5 is a schematic view of a label of the FIG. 3A illustrating a separation line between adjacent labels;

FIG. 6A is a sketch of the labels of the FIG. 3A shown in a tape roll configuration;

FIG. 6B is a sketch of one of the labels of FIG. 6A adhered to a syringe with the code extending from the syringe;

FIG. 6C is a sketch of the labels of FIG. 6A with a back side of one of the labels exposed;

FIG. 6D is a sketch of one of the labels of FIG. 6A adhered to a syringe with a portion of a back side of the label visible;

FIG. 7A is a sketch of the labels of the FIG. 3A shown in a precut roll configuration;

FIG. 7B is a sketch of one of the labels of FIG. 7A adhered to a syringe with the code extending from the syringe;

FIG. 8A is a sketch of a front side of another embodiment of label of the present invention having the code on the back side of the label;

FIG. 8B is a sketch of a back side of the label of FIG. 8A;

FIG. 8C is a front view of a strip of five of the labels of FIG. 8A;

FIG. 8D is a back view of the strip of labels of FIG. 8C;

FIG. 9A is a sketch of a label of the FIG. 8A shown in a tape roll configuration;

FIG. 9B is a sketch of one of the labels of FIG. 9A adhered to a syringe with the code extending from the syringe;

FIG. 10A is a sketch of a label of the FIG. 8A shown in a precut roll configuration;

FIG. 10B is a sketch of a one of the labels of FIG. 10A adhered to a syringe with the code extending from the syringe;

FIG. 11A is a sketch of a dispenser housing multiple rolls of the labels of FIG. 8A;

FIG. 11B is a schematic drawing of a portion of the dispenser of FIG. 13A showing a label cutter;

FIG. 12A is a sketch of another embodiment of a label of the present invention having two codes positioned adjacent to one another on a front side of the label;

FIG. 12B is a sketch of a back side of the label of FIG. 12A;

FIG. 12C is a sketch of the label of FIG. 12A shown standing on an edge thereof;

FIG. 12D is a sketch of the label of FIG. 12C shown with a bend between the two codes;

FIG. 12E is a sketch of the label of FIG. 12D shown with the back side portions of the code areas folded over onto and adhered to one another;

FIG. 13A is a schematic view of one embodiment for location of the two codes of the label of FIG. 12A;

FIG. 13B is a schematic view of another embodiment for location of the two codes of the label of FIG. 12A;

FIG. 14A is a sketch of the label of FIG. 12A shown adhered to a relatively large diameter syringe and showing one side of the label with the code thereon;

FIG. 14B is a sketch of the label of FIG. 12A shown adhered to a relatively small diameter syringe and showing one side of the label with the code thereon;

FIG. 15A is a sketch of the syringe of FIG. 14A showing the other side of the label with the code thereon;

FIG. 15B is a sketch of the syringe of FIG. 14B showing the other side of the label with the code thereon;

FIG. 16 is a sketch of a plurality of the labels of FIG. 12E shown in a tape roll configuration;

FIG. 17 is a sketch of a plurality of the labels of FIG. 12E shown in a precut roll configuration;

FIG. 18A is a schematic drawing of a preferred configuration for folding and winding a strip of the labels of FIG. 12A onto a tape roll configuration of FIG. 16;

FIG. 18B is a schematic drawing of a undesirable configuration for folding and winding a strip of the labels of FIG. 12A onto a tape roll configuration of FIG. 16;

FIG. 18C is a schematic drawing of one configuration for folding and winding a strip of the labels of FIG. 12A onto a precut roll configuration of FIG. 17;

FIG. 18D is a schematic drawing of another configuration for folding and winding a strip of the labels of FIG. 12A onto a precut roll configuration of FIG. 17;

FIG. 18E is a schematic drawing of another configuration for folding and winding a strip of the labels of FIG. 12A onto a precut roll configuration of FIG. 17;

FIG. 19 is a sketch of a syringe with a label of the present invention adhered thereto in an axial orientation;

FIG. 20 is a sketch of one of the labels of the present invention adhered to a relatively large diameter syringe and another of the labels adhered to tubing connected to the syringe;

FIG. 21A is a schematic drawing of a first step the labels of FIG. 16 being unwound from the tape roll wound in accordance with FIG. 18A;

FIG. 21B is a schematic drawing of a second step the labels of FIG. 16 being unwound from the tape roll wound in accordance with FIG. 18A;

FIG. 21C is a schematic drawing of a third step the labels of FIG. 16 being unwound from the tape roll wound in accordance with FIG. 18A;

FIG. 22A is a schematic drawing of a first step the labels of FIG. 16 being unwound from the tape roll wound in accordance with FIG. 18B;

FIG. 22B is a schematic drawing of a second step the labels of FIG. 16 being unwound from the tape roll wound in accordance with FIG. 18B; and

FIG. 22C is a schematic drawing of a third step the labels of FIG. 16 being unwound from the tape roll wound in accordance with FIG. 18B.

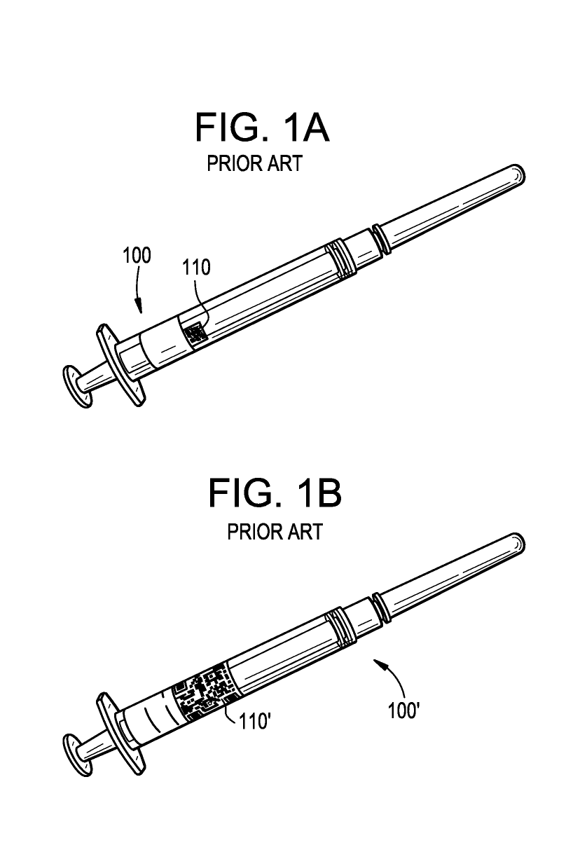

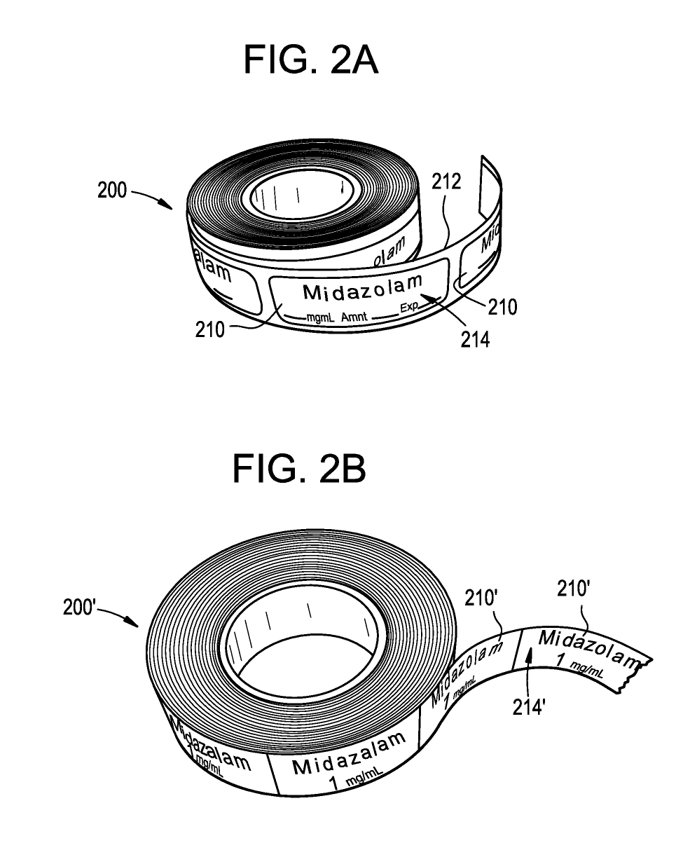

### DETAILED DESCRIPTION

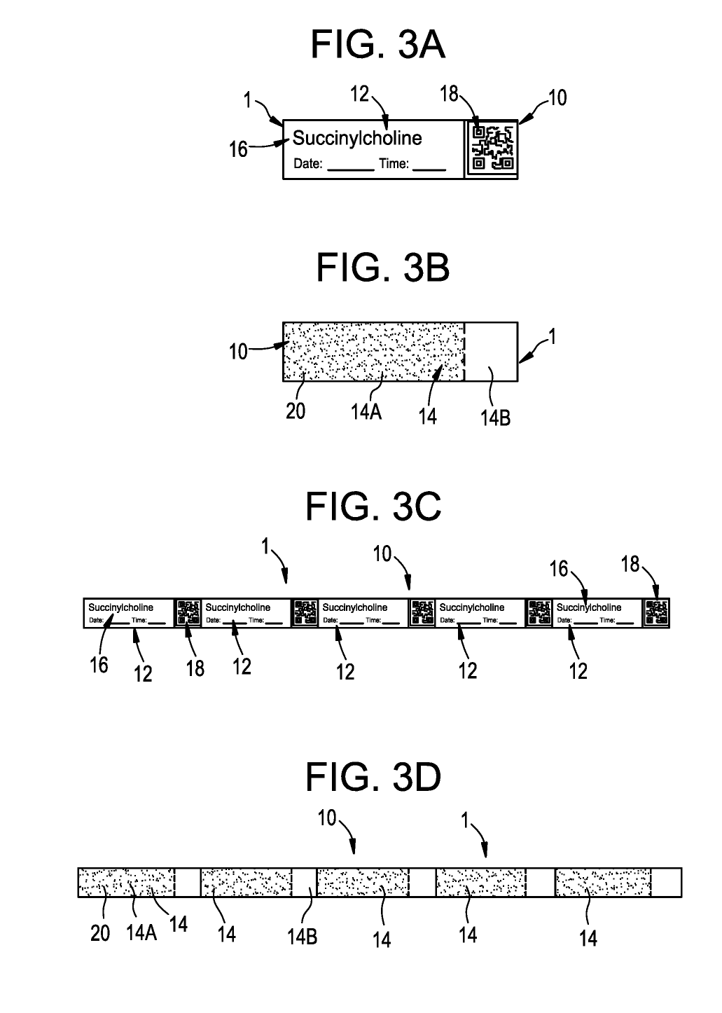

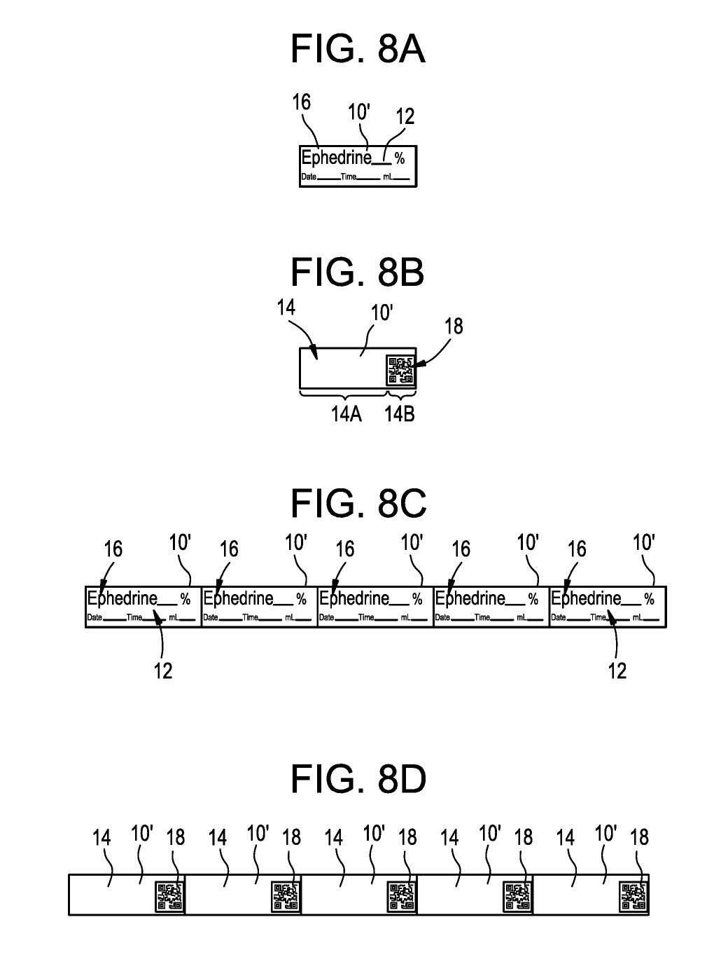

As shown in FIGS. 3A, 3B, 3C, and 3D, a labeling system for identification of an object such as a medication, a tube, a wire or a branch of a plant, is generally designated by the numeral 1. The labeling system 1 includes a label 10 having a first side 12 (e.g., front side) and a second side 14 (e.g., back side). The front side 12 defines a first indicia 16 of the object, such as but not limited to the name of the medication in text format (e.g., Succinylcholine), date and time. In one embodiment, the first indicia 16 is on a colored background. In one embodiment, the second indicia 18 is on a white background for better scanner reading, or can be on colored background, similar to the first side 12, for easy visual recognition the type of medication by a label users. Additional information related to the object (e.g., the medication) can also be shown on the front side 12 of the label 10 including but not limited to 1) the name of the drug therein contained, (2) the concentration of the drug therein contained, (3) the date and time that the syringe was prepared, (4) the name of the person who prepared the drug, and (5) the expiration date and time the drug was administered. The front side 12 also includes a second indicia 18 of the object (e.g., the medication), such as the matrix bar code (e.g., a QR Code®) as shown thereon. However, the second indicia 18 is not limited in this regard as other such indicia such as but not limited to linear bar codes, other scanable codes, magnetic strips and computer chip cards may be employed. While the second indicia 18 is described as being on the front side 12, the present invention is not limited in this regard as the second indicia may appear on the back side 14 as described further herein with regard to FIGS. 8A, 8B, 8C, 8D, 9A, 9B, 10A, and 10B.

As shown in FIG. 3B, the back side 14 has an adhesive 20 on a portion 14A thereof opposite the first indicia 16. The back side 14 has an area 14B free of adhesive. The area 14B free of adhesive is aligned with the second indicia 18.

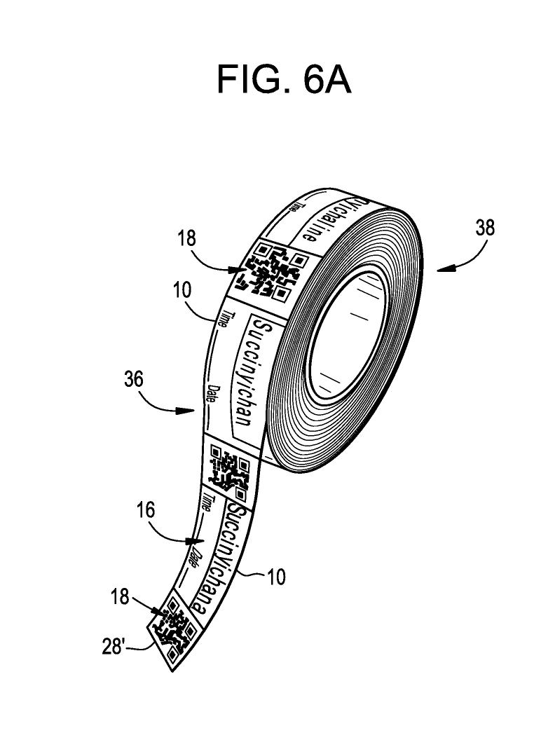

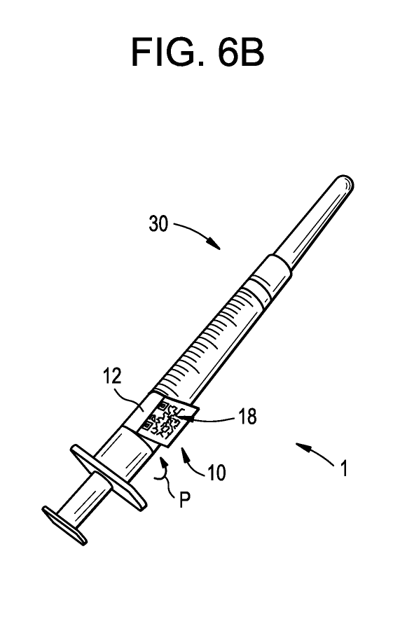

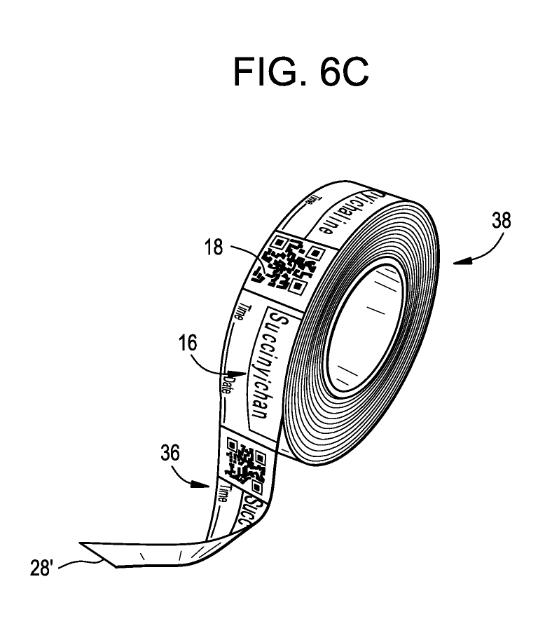

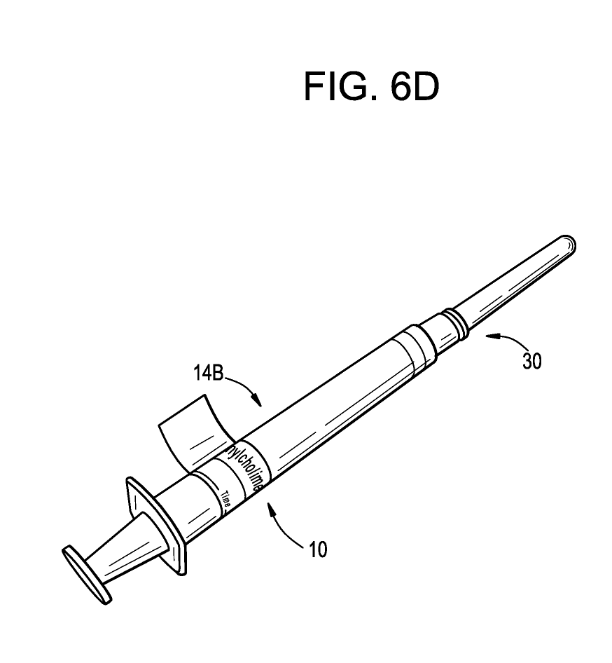

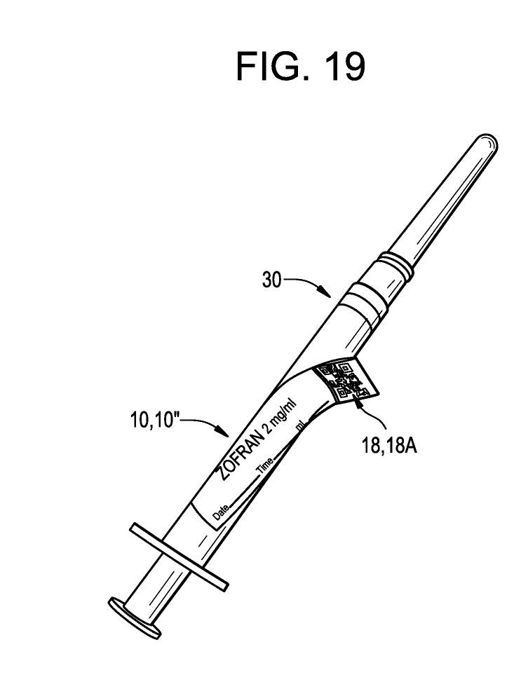

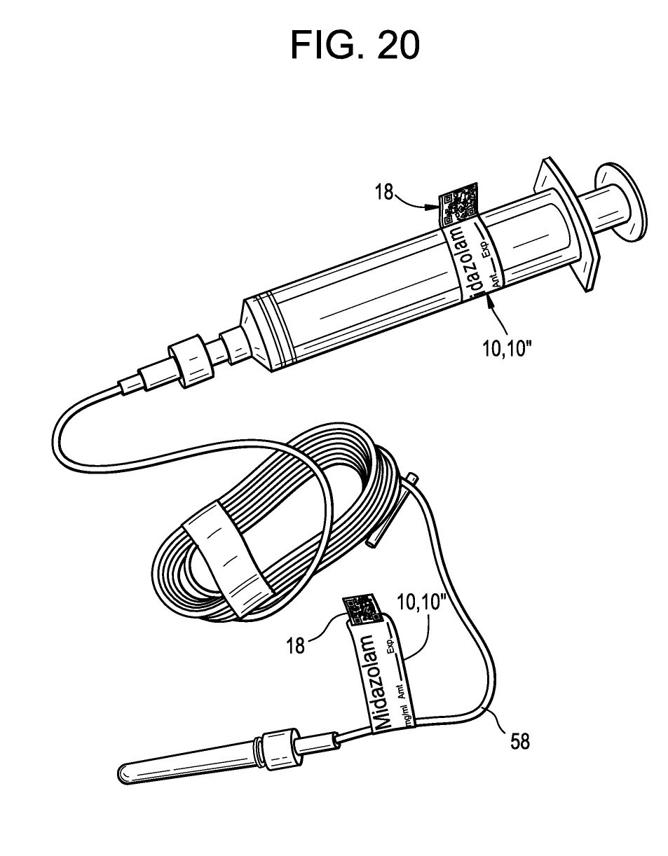

As shown in FIG. 6B, the labeling system 1 includes a dispenser for dispensing medication (e.g., a syringe 30) having one of the labels 10 adhered thereto. The adhesive 20 on the portion 14A of the back side 14 of the label 10 is circumferentially wrapped around the syringe 30. The area 14B free of adhesive protrudes outwardly from the syringe 30 so that the second indicia 18 is in a generally planar form to facilitate reading by a scanner (not shown). The portion 14B of the label 10 has no adhesive thereon and therefore does not stick as shown in FIGS. 6B and 6D. While the label 10 is described as being circumferentially wrapped around the syringe 30, the present invention is not limited in this regard as the label 10 may be secured axially to the syringe as shown in FIG. 19 or secured to tubing 50 as shown in FIG. 20.

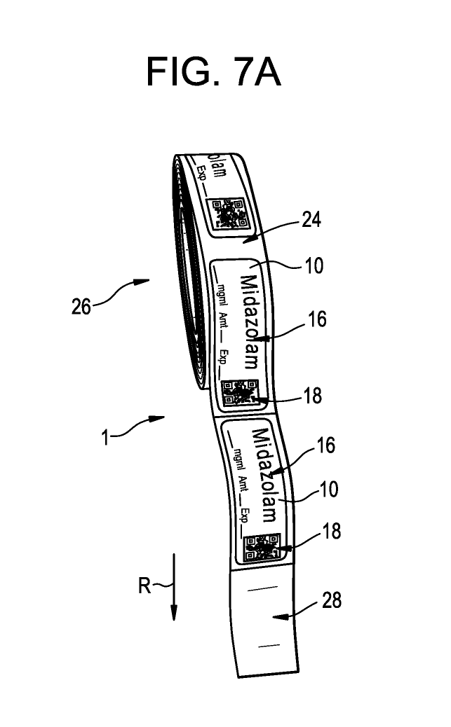

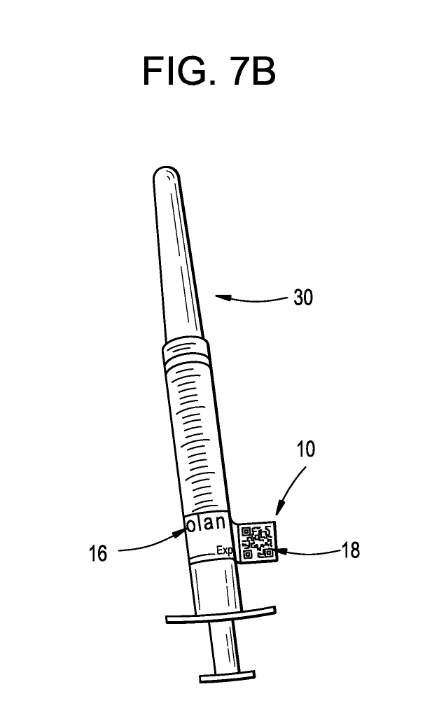

As shown in FIG. 7A, a plurality of the labels 10 are spaced apart from one another and releaseably disposed on a length of a release substrate 24. The release substrate 24 is manufactured from any suitable material such as siliconized papers or films. In the embodiment illustrated in FIG. 7A, the length of the release substrate 24 is in a precut roll configuration 26 wherein individual labels 10 are releaseably adhered to the release substrate so that the labels 10 are separate from and spaced apart from one another. The labels 10 may be peeled off of the release substrate 24. After removal of the label 10 from the release substrate 24, the label 10 may be adhered to the syringe 30 as shown in FIG. 7B and as described herein with regard to FIG. 6B.

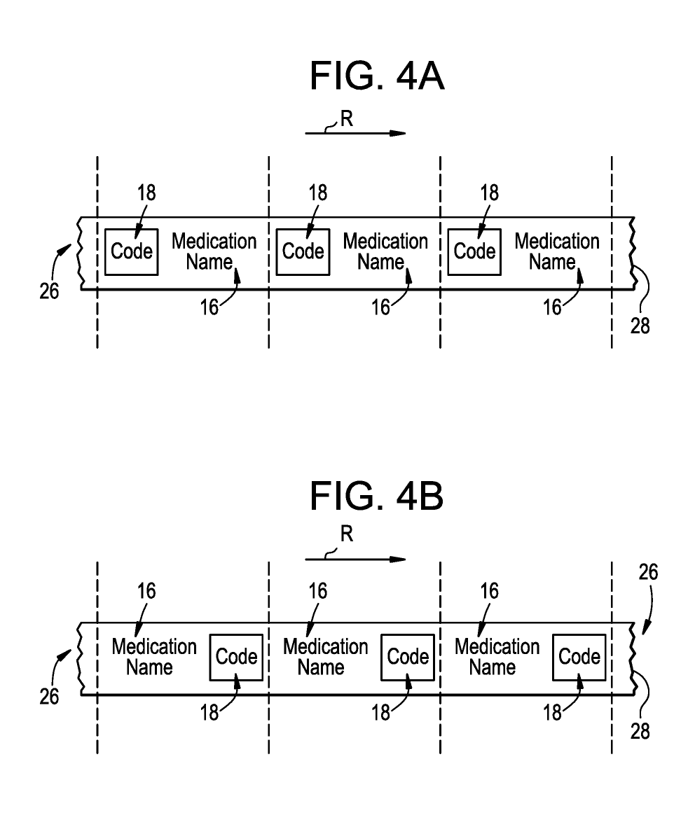

As shown in FIG. 7A, in the precut roll configuration 26 the release substrate 24 defines a dispensing edge 28 which can be used to facilitate unrolling the labels 10 from the precut roll 26 in the direction indicated by the arrow R. In one embodiment, as shown in FIGS. 4B and 7A, the area 14B free of adhesive and the second indicia 18 are positioned in a direction facing the dispensing edge 28. While the area 14B free of adhesive and the second indicia 18 are shown and described as being positioned in a direction facing the dispensing edge 28, the present invention is not limited in this regard as the first indicia 16 is positioned in a direction facing the dispensing edge 28 as shown in FIG. 4A.

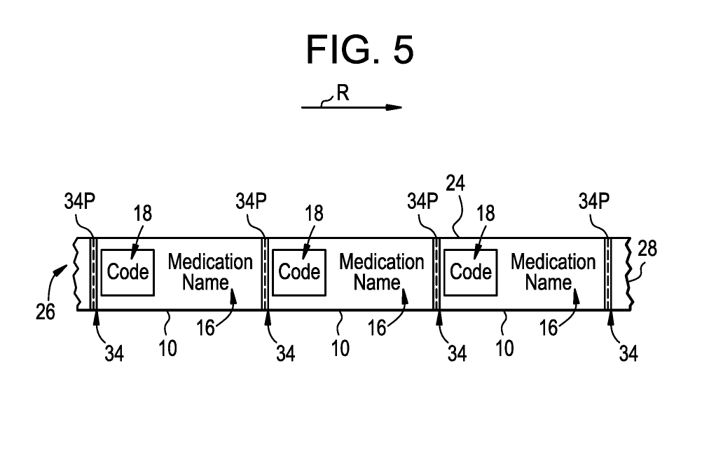

As shown in FIG. 5, the release substrate 24 includes a separation or demarcation line 34 between adjacent labels 10 for locating a cutting position. In one embodiment, the separation or demarcation line 34 includes a line of perforations 34P extending through the release substrate 24 between adjacent labels 10 for severing adjacent portions of the release substrate 24 from one another.

As shown in FIGS. 3C, 3D, 8C, 8D and 9A, the labels 10 are connected to one another in a continuous tape configuration 36. As shown in FIG. 6A, the continuous tape configuration is in a roll 38. In the continuous tape configuration 36, the labels 10 are connected directly to one another. The roll 38 has a dispensing edge 28′ as shown in FIG. 6A. In one embodiment, the area 14B free of adhesive and the second indicia are positioned facing the dispensing edge 28′. However, the present invention is not limited in this regard, as the first indicia 16 may be positioned facing the dispensing edge 28′ similar to that shown in FIG. 4A for the roll 26.

In one embodiment, the labels 10 in the tape configuration 36 include a separation or demarcation line 34 between adjacent labels 10 for locating a cutting position, similar to that shown and described for the roll 26 with reference to FIG. 5. In one embodiment, the separation or demarcation line 34 includes a line of perforations 34P extending through the tape configuration 36 between adjacent labels 10 for severing adjacent portions of the tape configuration from one another, similar to that described above for the roll 26 with reference to FIG. 5.

The demarcation line 34 is of such a width that it is spaced sufficiently apart from the second indicia 18, so that the second indicia 18 remains intact when the label 10 is separated from the roll 26. When the demarcation line is narrow, it is more likely that the second indicia 18 will be damaged during separation, due to the increased difficulty of aligning the demarcation line 34 with the cutting edge. When the demarcation line 34 is wider, it is less likely that the second indicia 18 will be damaged during separation. However, a wider demarcation line 34 limits the number of labels 10 that can fit on a roll 26. In one embodiment, the demarcation line 34 is about 1.0 mm. (0.039 inches) wide. In one embodiment, the demarcation line 34 is about 2.0 mm. (0.078 inches) wide. In one embodiment, the demarcation line 34 is about 1.0 cm. (0.39 inches) wide. In one embodiment, the demarcation line 34 is up to about 2.0 cm. (0.78 inches) wide. In one embodiment, the demarcation line 34 is of a different color than other portions of the label 10 to facilitate visual recognition of the demarcation line 34.

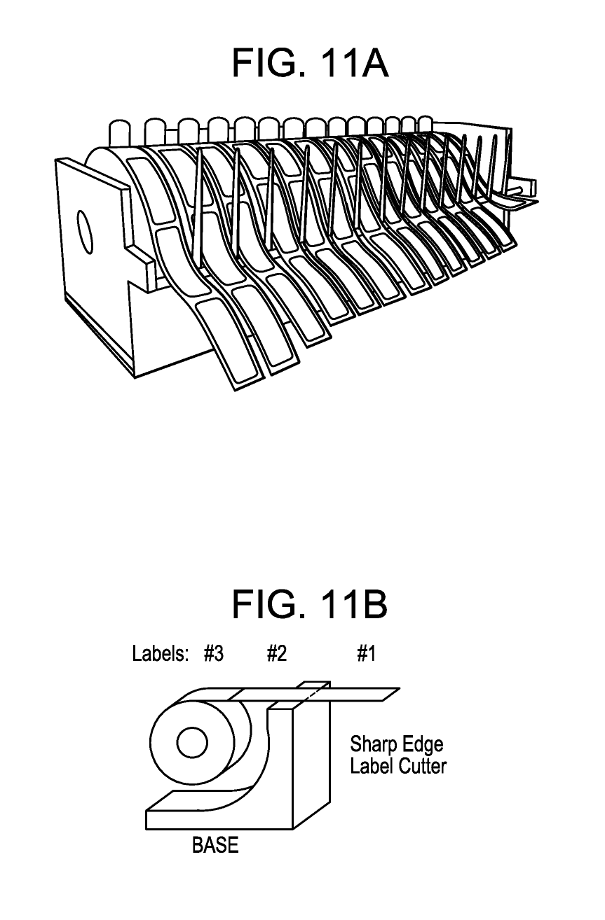

In one embodiment, as shown in FIG. 11B, the second indicia 18 includes a rip resistant and/or cut resistant material thereon. The rip resistant and/or cut resistant material is configured to prevent damage to the second indicia 18 upon separation of the label 10 from the roll 26.

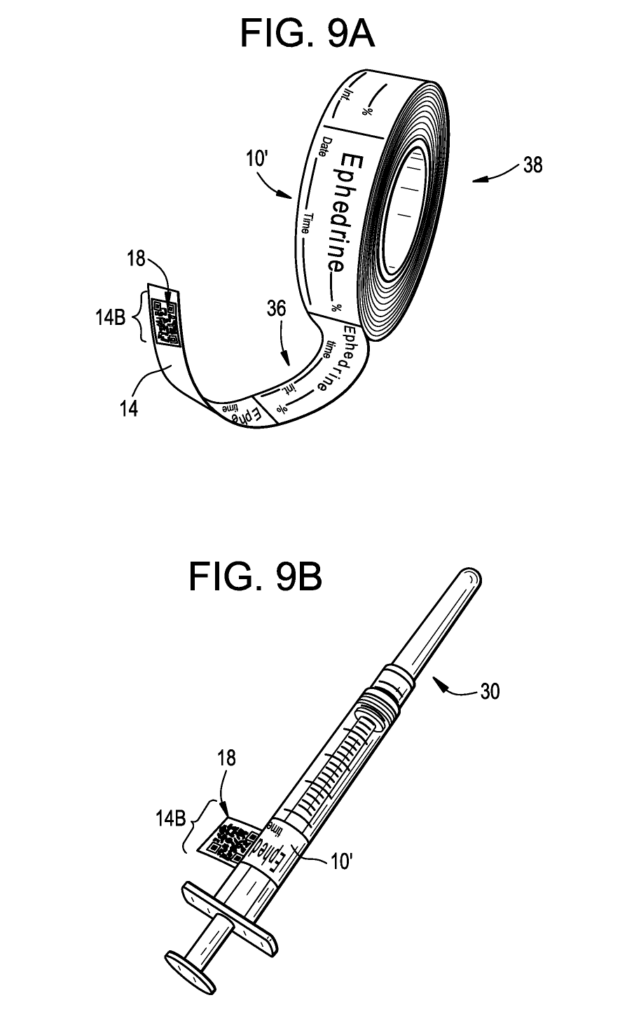

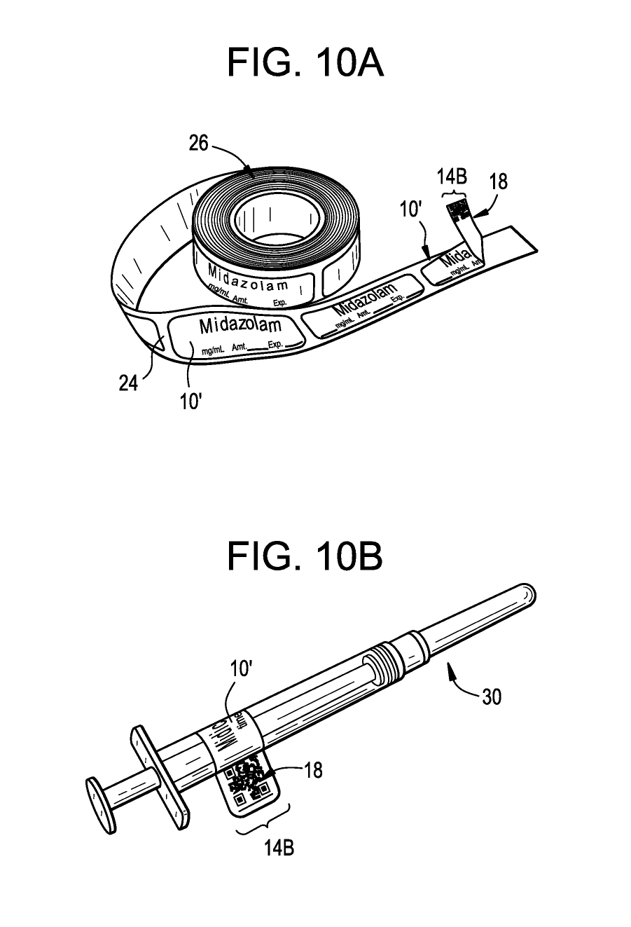

The label 10′ shown in FIGS. 8A, 8B, 8C, 8D, 9A and 10A is similar to the label 10 shown in FIGS. 3A, 3B, 3C, 3D, 6A, 6C and 7A, with the notable exception that the second indicia 18 is positioned on the back side 14 of the label 10′ in the area 14B free of adhesive. As shown in FIG. 9A, the labels 10′ are configured in a continuous tape configuration 36 in a roll 38. As shown in FIG. 10A, the labels 10′ are disposed on the releasable substrate 24 in a continuous precut roll 26. As shown in FIGS. 9B and 10B, the labels 10′ are adhered to the syringe 30 as described herein with regard to FIG. 6B, with the notable exception that the second indicia 18 appears in the area 14B free of adhesive on the back side 14 of the label 10′ of FIGS. 9A and 10A, respectively.

In the embodiment shown in FIGS. 8A, 8B, 8C, 8D, and 10A, the second indicia 18 is prevented from being scanned accidentally while in the roll 38 by the first side 12 of the label. In this embodiment, the second indicia 18—being positioned on the back side 14 of the label 10′ and consequently facing the roll 38—is not exposed, and is therefore incapable of being read by a so-configured device. Thus, the label 10′ according to this embodiment is configured to allow the second indicia 18 to be read by a compatible device only once the label 10′ is removed from the roll 38 and adhered to the syringe 30.

In one embodiment, the first indicia 16 includes different material than the second indicia 18. For example, the second indicia 18 includes high quality paper and the first indicia 16 includes low quality paper. The first indicia 16 and the second indicia 18 are configured on separate continuous rolls 38. The second indicia 18 is removed from the roll 38 and adhered to the first indicia 16. This embodiment is similar to that shown in FIGS. 3A, 3B, 3C, 3D, 8A, 8B, 8C, 8D, and 10A.

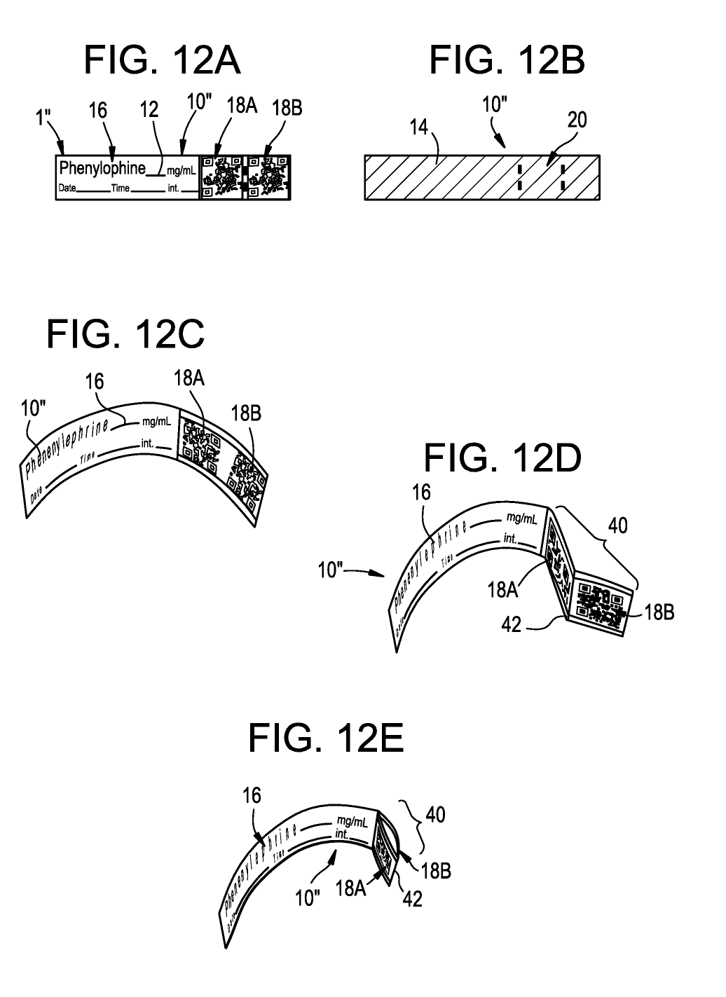

As shown in FIGS. 12A, 12B, 12C, 12D and 12E, another embodiment of the labeling system is designated by the numeral 1″. The labeling system 1″ includes a label 10″ having a first side 12 (e.g., front side) and a second side (e.g., back side). The front side 12 includes a first indicia 16 of the medication and two second indicia 18A and 18B of the medication positioned adjacent to one another. The first indicia 16 and the two second indicia 18A and 18B are similar to the first indicia 16 and the second indicia 18, described herein with regard to FIGS. 3A and 3B, respectfully. However, essentially all of the second side 14 has the adhesive applied thereto, including the portion of the back side 14 that is aligned with the second indicia 18A and 18B.

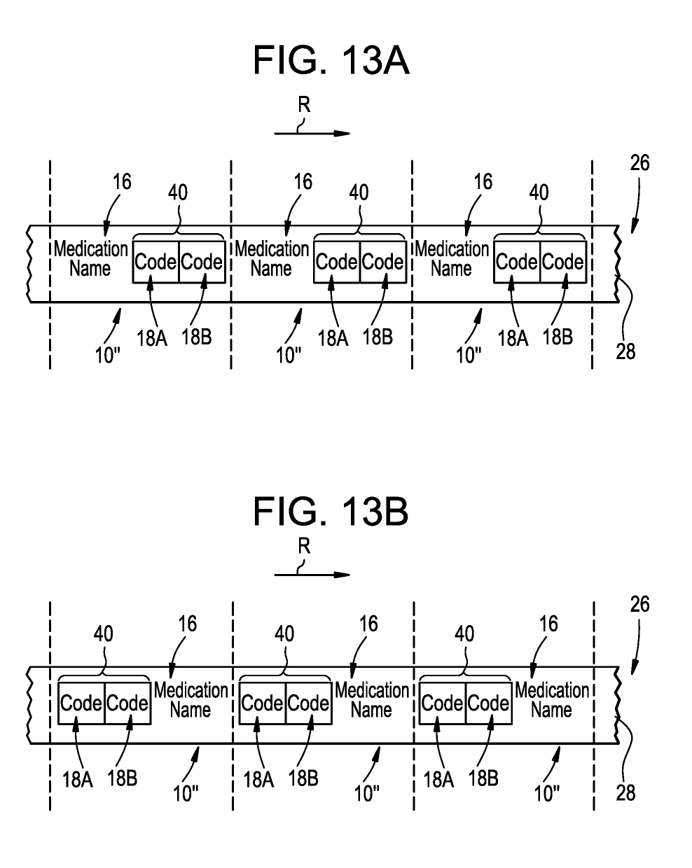

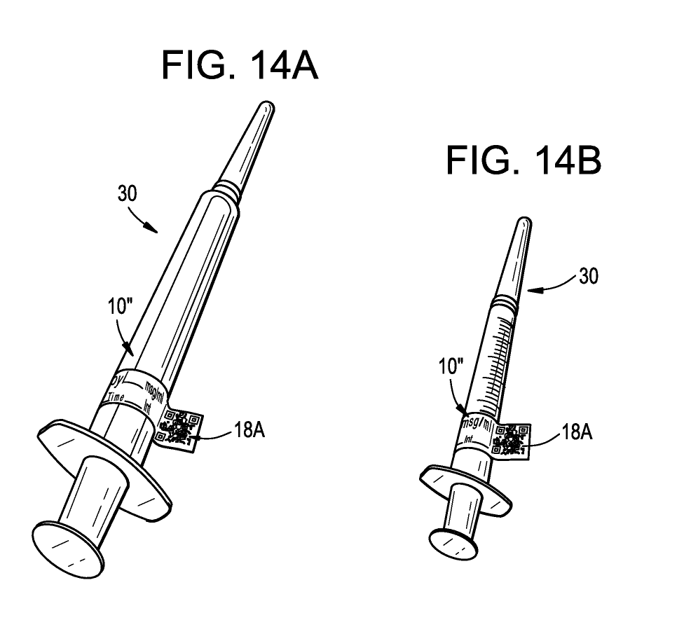

As shown in FIG. 13A, the second indicia 18A and 18B are positioned proximate an end of the label 10″ facing away from the dispensing end 28. As shown in FIG. 13B, the second indicia 18A and 18B are positioned proximate an end of the label 10″ facing away from the dispensing end 28.

As shown in FIGS. 12D and 12E, the label 10″ has a folded over segment 40 in which the label 10″ is folded along an axis 42 between the two second indicia 18A and 18B so that portions of the back surface 14 aligned with the second indicia 18A and 18B adhere to one another so that the two second indicia 18A and 18B are facing outward.

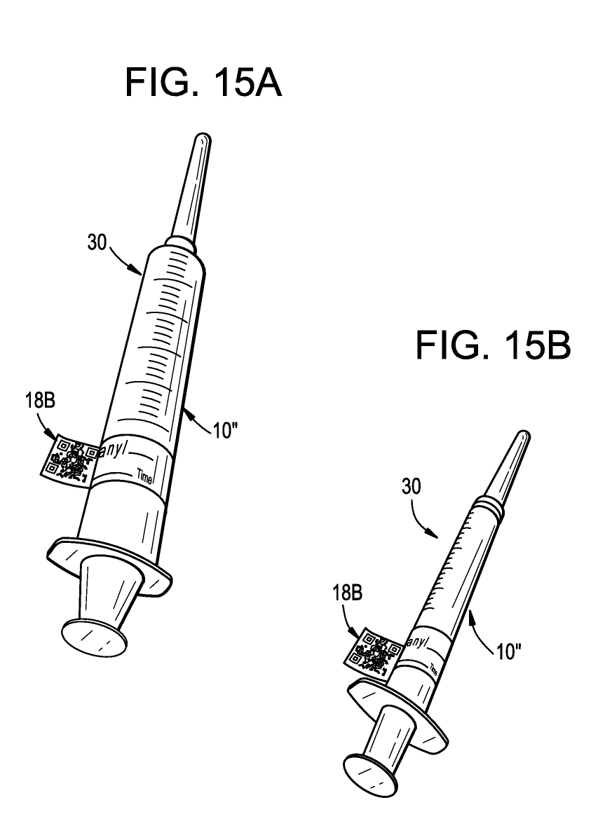

As shown in FIG. 14A, the label 10″ is adhered to the large diameter syringe 30 so that the second indicia 18A extends away from the syringe 30 and is scanable. As shown in FIG. 14B, the label 10″ is adhered to the small diameter syringe 30 so that the second indicia 18A extends away from the syringe 30 and is scanable. As shown in FIG. 15A, the large diameter syringe 30 is rotated 180 degrees along a longitudinal axis relative to the position of the syringe in FIG. 14A. In FIG. 15A, the second indicia 18B extends away from the syringe 30 and is scanable. As shown in FIG. 15B, the small diameter syringe 30 is rotated 180 degrees along a longitudinal axis relative to the position of the syringe in FIG. 14B. In FIG. 15B, the second indicia 18B extends away from the syringe 30 and is scanable.

As shown in FIG. 17, a plurality of the labels 10″ are spaced apart from one another and disposed on a length of a release substrate 24. The length of the release substrate 24 is in a roll 26 configuration and has a dispensing edge 28. The two second indicia 18A and 18B are positioned facing the dispensing edge 28. As shown in FIG. 17, the folded over segment 40 is positioned at an end of the label 10″ facing away from the dispensing edge 28.

In one embodiment, the labeling system 1″ includes a line of separation or demarcation and/or a line of perforations extending through the release substrate 24 between adjacent labels 10″ for severing the release substrate 24, similar to that described herein with regard to FIG. 5.

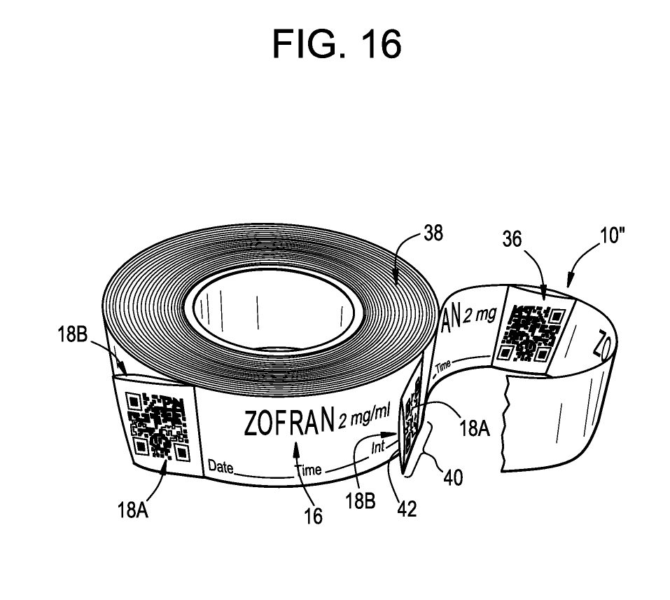

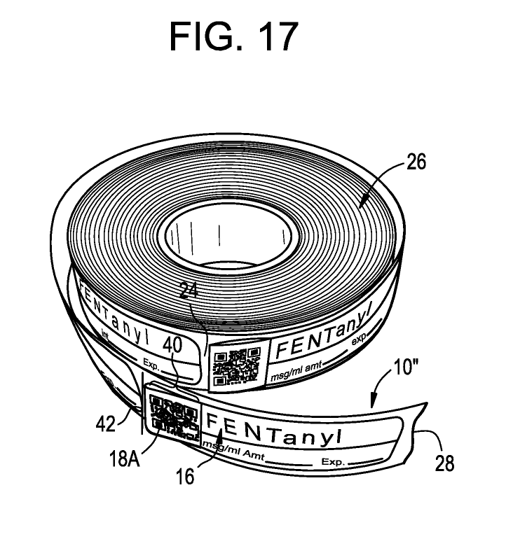

As shown in FIG. 16, a plurality of the label 10″ are connected to one another in a continuous tape configuration 36 and further configured in a roll configuration 38.

In one embodiment, the folded over segment 40 is positioned proximate the dispensing edge 28 and has a free end facing away from the dispensing edge 28.

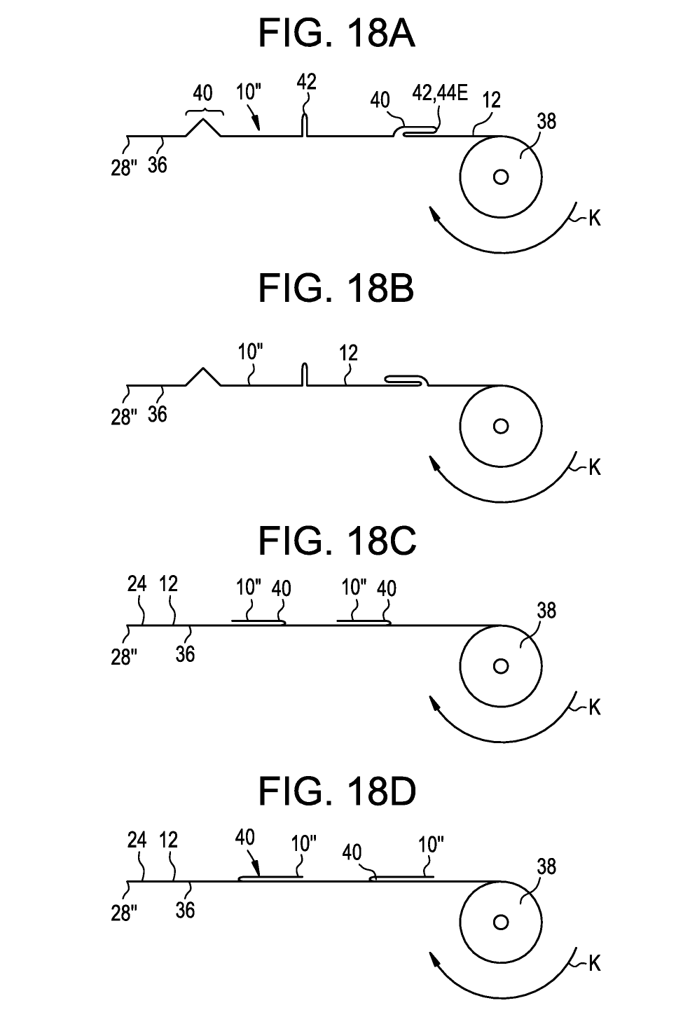

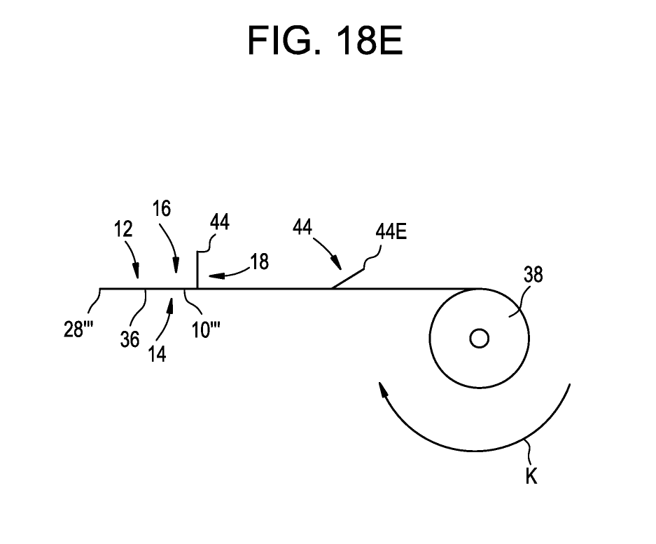

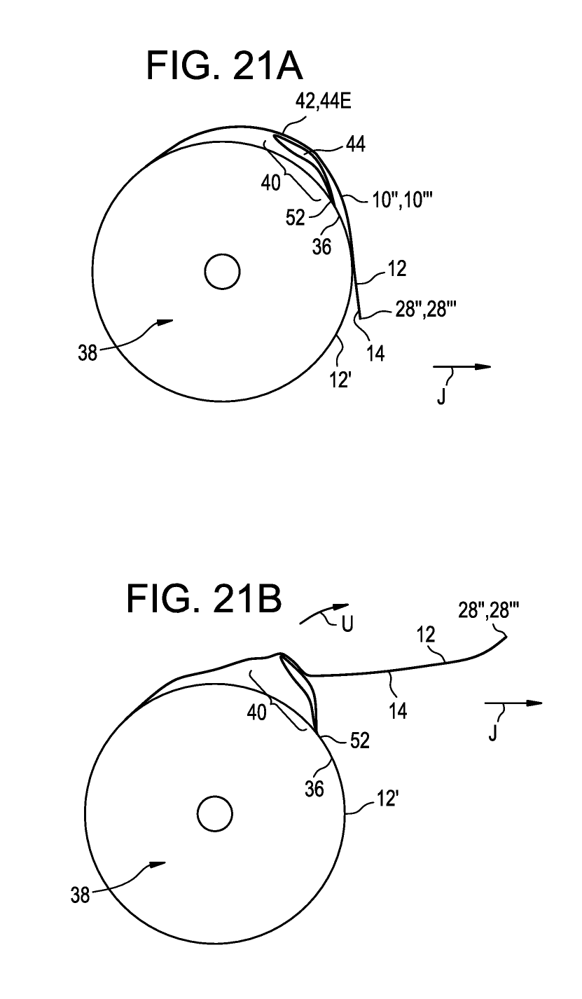

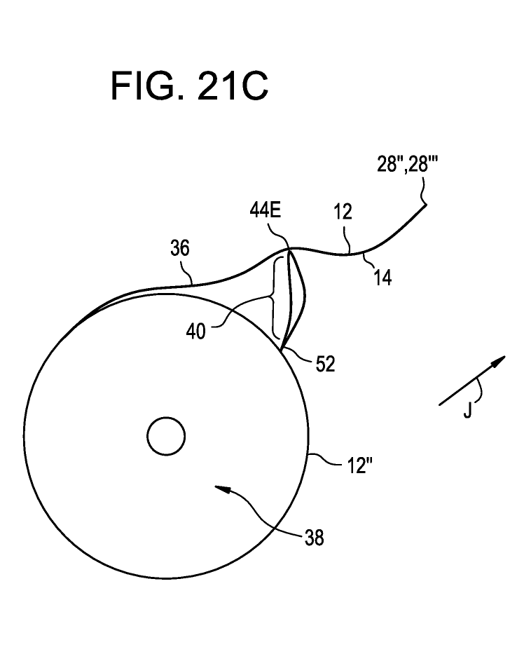

As shown in FIG. 18E, the labeling system 1′″ for identification of a medication includes a plurality of labels 10′″ connected to one another in a continuous tape configuration 36. The continuous tape configuration 36 is in a roll configuration 38. The continuous tape configuration 36 defines a dispensing edge 28′″ extending away from the roll configuration 38. The labels 10′″ have a front side 12 and a back side 14. The front side 12 has a first indicia 16 of the medication thereon. The first side 12 has an extension segment 44 extending therefrom. The extension segment 44 has a second indicia 18 of the medication positioned thereon. The back side 14 has an adhesive thereon. The extension segment 44 has a free end 44E facing away from the dispensing edge 28′″. When the tape configuration 36 is wound onto the roll configuration 38, the extensions 44 are pressed flat against adjacent layers of the labels 10′″ with the free end 44E facing away from the free end 28′″ which facilitates unwinding of the labels 10′″ as described herein with reference to FIGS. 18A, 21A, 21B and 21C.

As shown in FIG. 18A in a preferred configuration for rolling labels 10″ of a continuous tape configuration 36, the roll 38 is rotated in the direction of the arrow K and the folded over segments 40 are laid flat against the front side 12 of the tape 36 with the free end 44E of the folded over segment 40 facing away from the dispensing end and towards the roll 38. As a result of testing and experimentation, the inventors surprisingly found that rolling the labels 10″ onto the roll 38 with the free end 44E facing away from the dispensing end and towards the roll 38 facilitated unrolling the labels 10″ from the roll 38. In particular, with reference to FIG. 21A as the dispensing end 28″, 28′″ of the continuous tape configuration 36 is pulled away from the roll 38 in the direction of the arrow J the back side 14 peels away from the top side 12′ that the back side 14 is adhered to in the roll 38. As shown in FIG. 21B, as the dispensing end 28″, 28′″ is pulled further away from the roll 38, the back side 14 peels away from the folded over segment 40 thereby lifting the folded over segment 40 away from the roll 38 while the folded over segment remains anchored to the tape 36 at an anchor point 52. The back side 14 can begin to peel away from the folded over segment 40 at the anchor point 52 because the anchor point 52 secures the folded over segment 40 the tape 36. As shown in FIG. 21C, as the dispensing end 28″, 28′″ is pulled further in the direction indicated by the arrow J, the back side 14 peels entirely off of the folded over segment 40 as the back side 14 peels away from the free end 44E.

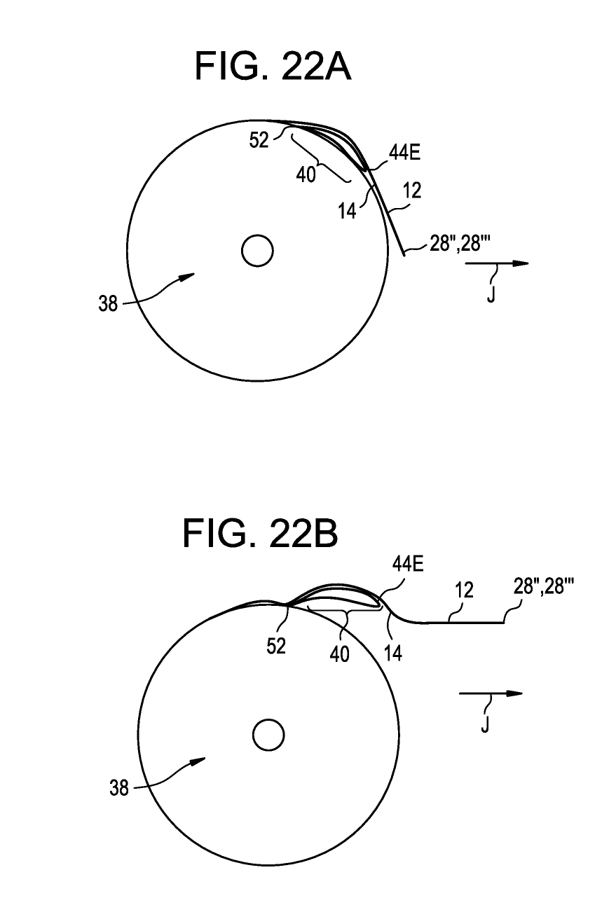

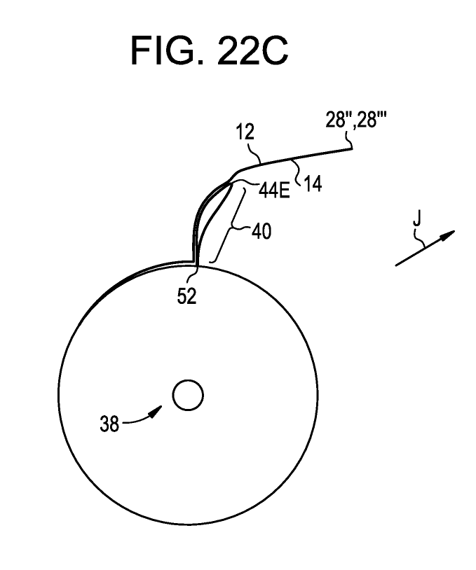

In contrast, the inventors have found through testing and experimentation that if the folded over segments 40 are laid flat against the tape 36 with the free end 44E facing the dispensing end 28 and away from the roll 38 as shown in FIG. 18B, the tape cannot be readily unrolled from the roll 38 because the bottom side 14 of the tape 36 does not peel away from the folded over segment 40 as shown in FIGS. 22A, 22B and 22C. As shown in FIG. 22A, the back side 14 is adhered to the folded over segment 40 as the dispensing end 28″, 28′ is pulled away from the roll 38 in the direction of the arrow J. As shown in FIG. 22B, as the dispensing end 28″, 28′″ is pulled further away from the roll 38 the back side 14 remains adhered to the folded over segment 40 and lifts the folded over segment away from the roll 38. As shown in FIG. 22C, continued pulling of the dispensing end 28″, 28′″ in the direction of the arrow J does not peel the back side 14 off of the folded over segment 40, but instead further pulls the folded over segment 40 away from the roll 38.

As shown in FIG. 18C, in the configuration for rolling labels 10″ disposed on the release substrate 24 in the precut tape configuration 36, the roll 38 is rotated in the direction of the arrow K and the folded over segments 40 are laid flat against the release substrate 24 of the tape 36 with the free end 44E of the folded over segment 40 facing away from the dispensing end 28 and towards the roll 38.

As shown in FIG. 18D, in the configuration for rolling labels 10″ disposed on the release substrate 24 in the precut tape configuration 36, the roll 38 is rotated in the direction of the arrow K and the folded over segments 40 are laid flat against the release substrate 24 of the tape 36 with the free end 44E of the folded over segment 40 facing the dispensing end 28 and away from the roll 38. The labels 10″ disposed on the release substrate 24 in the precut tape configuration 36 can readily be unrolled without adherence of the folded over segment 40 to the release layer for rolls 36 that that have been rolled in accordance with FIG. 18C or FIG. 18D.

Although the labeling system 1 is shown and described in reference to labeling medication, the present invention is not limited in this regard, as it will be understood that the labeling system 1 described herein may be employed for any suitable purpose to the same effect without departure from the broader aspects of the present invention, including, but not limited to, labels for use in identifying objects such as tubes, wires or branches of plants.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A labeling system for identification of an object, the labeling system comprising:
at least one label having a first side and a second side;
the first side defining a first indicia of the object and at least two second indicia of the object positioned adjacent to one another;
the second side having an adhesive thereon;
wherein a portion of the at least one label has a folded over segment in which the at least one label is folded along an axis between the at least two second indicia so that portions of the second side surface aligned with the second indicia adhere to one another so that the at least two second indicia are facing outward.

2. The labeling system of claim 1, wherein a plurality of the at least one label are connected to one another in a continuous tape configuration.

3. The labeling system of claim 1, wherein the folded over segment has a free end facing away from the dispensing edge.
